(19) United States
(12) Patent Application Publication
(10) Pub. No.: US 2023/0405148 A1
(43) Pub. Date: Dec. 21, 2023

(54) GENE THERAPY FOR ALZHEIMER'S DISEASE

(71) Applicant: Cornell University, Ithaca, NY (US)

(72) Inventors: Ronald G. Crystal et al., New York, NY (US); Katie M Stiles, Bronx, NY (US)

(21) Appl. No.: 17/769,255

(22) PCT Filed: Oct. 16, 2020
(86) PCT No.: PCT/US2020/056051

§ 371 Date: Apr. 14, 2022

Related U.S. Application Data
(60) Provisional application No. 62/915,988, filed on Oct. 16, 2019.

(51) Int. Cl. A61K 48/00; C12N 15/113; C07K 14/775

(52) U.S. Cl. CPC A61K 48/005; C12N 15/113; C07K 14/775; C12N 2310/531; C12N 2310/14; C12N 2750/14143; C12N 2310/141; C12N 2320/31

(57) ABSTRACT

Compositions and methods to prevent, inhibit or treat a disease or disorder associated with expression of APOE4 in a mammal are provided.

### CROSS-REFERENCE TO RELATED APPLICATIONS

This application claims the benefit of the filing date of U.S. application No. 62/915,988, filed on Oct. 16, 2019, the disclosure of which is incorporated by reference herein.

### BACKGROUND

Apolipoprotein E (APOE) is an important central nervous system (CNS) apolipoprotein intimately involved in the pathogenesis of the most common late-onset familial and sporadic forms of Alzheimer's disease (AD; Yu et al., 2014). In the general population, there are 3 common APOE alleles (ε4, ε3, and ε2) that encode the 3 APOE isoforms expressed primarily in the liver and brain. APOE4 carriers have a markedly increased risk of developing AD (3-15 fold for beterozygotes and homozygotes, respectively, compared with APOE3 homozygotes) and an earlier age-of-onset for developing the disease (approximately 5 years for each 84 allele; Corder et al., 1993; Farrer et al., 1997; Lambert et al., 2013; Saunders et al., 1993; Strittmatter et al., 1993). The fact that 45% of AD patients carry at least 1 ε4 allele (compared with only 15% of age-matched healthy controls) makes APOE4 by far the most common genetic risk factor for late-onset AD, the most common form of AD. By contrast, APOE2 is a protective allele reducing AD risk by approximately 50% and markedly delaying the age-of-onset (Corder et al., 1994; Farrer et al., 1997; Suri et al., 2013: Talbot et al., 1994; Yu et al., 2014).

The major physiological differences between APOE3, the most common isoform, and APOE2 and APOE4, are due to differences in amino acids at 1 of 2 positions, residues 112 (APOE4) and 158 (APOE2), which are cysteine-arginine interchanges (Hatters et al., 2006). Differences in these 2 amino acids result in differences in protein structure, and the corresponding binding affinities of these APOE isoforms to lipoproteins, lipoprotein receptors, and in regulating A1 aggregation, degradation, efflux, and phagocytosis (Castellano et al., 2011; Deane et al., 2008; Hashimoto et al., 2012; Hatters et al., 2006; Holtzman et al., 2012; Li et al., 2012; Manelli et al. 2004; Walker et al., 2000; Yu et al., 2014; Zhao et al., 2009).

### SUMMARY

In one embodiment, the present disclosure provides a gene therapy vector for Alzheimer's disease. In one embodiment, a gene therapy vector comprises an AAV expression vector encoding the human APOE2 gene and either in cis or in trans artificial microRNA(s) that target endogenous APOE4. This vector system silences the expression of detrimental endogenous APOE4 in combination with supplementation of the beneficial APOE2 gene from a gene therapy vector, e.g., an AAV vector. As disclosed herein, exemplary artificial microRNA sequences were designed that target the endogenous APOE4 mRNA for suppression. The microRNAs (miRNAs) may be incorporated in sequences that are 5′ to the APOE2 coding sequence, e.g., in a n intron such as the CAG promoter intron, or sequences that are 3′ to the APOE2 coding sequence, e.g., sequences that are 5′ to the polyA tail of the vector transgene plasmid coding for the human APOE2 coding sequence. Alternatively, the microRNA(s) may be inserted between a PolIII promoter, e.g., a U6 promoter, and a terminator following the polyA site of the APOE2 expression cassette. The vector-derived human APOE2 DNA sequence optionally includes silent nucleotide changes to decrease or inhibit suppression by the microRNAs and in one embodiment may include a tag such as a HA tag for detection, e.g., for pre-clinical detection studies. In one embodiment, the expression construct is packaged into an AAV capsid of a serotype that targets astrocytes and glial cells (for example AAV9) the prominent sites of endogenous APOE expression in the CNS, but can be provided in other vectors, e.g., other viral vectors, plasmids, nanoparticle or liposomes.

In one embodiment, a gene therapy vector is provided comprising a first promoter operably linked to a nucleic acid sequence comprising an open reading frame encoding APOE2 and a 3′ untranslated region, and an isolated nucleotide sequence is provided comprising one or more RNAi nucleic acid sequences for inhibition of APOE4 mRNA. In one embodiment, the vector comprises the nucleotide sequence. In one embodiment, the nucleotide sequence is inserted 5′ or 3′ to the open reading frame. In one embodiment, the nucleotide sequence is inserted 5′ and 3′ to the open reading frame. In one embodiment, the nucleotide sequence is on a different vector. In one embodiment, the isolated nucleotide sequence comprises a second promoter operably linked to the one or more RNAi nucleic acid sequences. In one embodiment, the gene therapy vector is a viral vector. In one embodiment, the different vector is a viral vector. In one embodiment, the viral vector is an AAV, adenovirus, lentivirus, herpesvirus or retrovirus vector. In one embodiment, the AAV is AAV5, AAV9 or AAVrh10. In one embodiment, the APOE4 is human APOE4. In one embodiment, the APOE2 is human APOE2. In one embodiment, the first promoter is a PolI promoter. e.g., a constitutive promoter or a regulatable promoter, for example, an inducible promoter. In one embodiment, the second promoter is a PolIII promoter. In one embodiment,

the isolated nucleotide sequence comprises nucleic acid for one or more miRNA comprising two or more of the RNAi nucleic acid sequences, e.g., one or more RNAi sequences are embedded in a miRNA sequence. In one embodiment, the RNAi comprises siRNA including a plurality of siRNA sequences. In one embodiment, the RNAi comprises shRNA sequences of about 15 to 25 nucleotides in length. In one embodiment, the open reading frame for APOE2 comprises a plurality of silent nucleotide substitutions relative to SEQ ID NO:6. e.g., the open reading frame comprises SEQ ID NO:7 or nucleotide sequence with at least 70%, 75% 80%, 85%, 90%, 95%, 97% or 98% nucleic acid sequence identity to SEQ ID NO:7 and encodes APOE2, or the open reading frame encodes APOE2 and comprises a nucleotide sequence with at least 70%, 75% 80%, 85%, 90%, 95%, 97% or 98% nucleic acid sequence identity to GAAAGAACTCAAAGCTTATA AGAGCGAGCTGGAGG (SEQ ID NO:13) but which sequence is not SEQ ID NO:7. In one embodiment, the plurality of the silent nucleotide substitutions in the APOE2 open reading frame are not in the RNAi nucleic acid sequence in the isolated nucleotide sequence, that is the sequence with the nucleotide substitutions differs from the RNAi nucleotide sequence so that the mRNA having the nucleotide substitutions does not bind to, e.g., for a duplex with, the RNAi sequences, e.g., isolated RNAi or RNAi sequences expressed from a vector. In one embodiment, at least 50%, 60%, 70%, 80% or 90% of codons in the open reading frame for APOE2 have a silent nucleotide substitution. In one embodiment, at least 5%, 10%, 20%, 30%, or 40%, of codons in the open reading frame for APOE2 have a silent nucleotide substitution, e.g., in a portion of APOE2 sequences that correspond to the RNAi sequences. That is, the silent nucleotide substitutions in a human APOE2 coding sequence result in a sequence that differs from endogenous human APOE4 sequences and differs from the APOE4 RNAi sequences. In one embodiment, the APOE4 that is inhibited has a sequence having at least 80%, 85%, 90%, 95% or more amino acid sequence identity to a polypeptide encoded by SEQ ID NO:22. In one embodiment, the APOE2 has a sequence having at least 80%, 85%, 90%, 95% or more amino acid sequence identity to a polypeptide encoded by SEQ ID NO:9. In one embodiment, the one or more RNAi nucleic acid sequences have at least 60%, 70%, 80%, 90% or more nucleotide sequence identity to one of SEQ ID Nos. 1-4 or 20-22 or the complement thereof. In one embodiment, the vector has a first PolI promoter operably linked to a nucleic acid sequence comprising an open reading frame encoding human APOE2 and an isolated nucleotide sequence having one or more RNAi nucleic acid sequences for inhibition of human APOE4 mRNA. In one embodiment, the nucleotide sequence is inserted 5′ to the open reading frame. In one embodiment, the nucleotide sequence is inserted 3′ to the open reading frame. In one embodiment, the nucleotide sequence is inserted 5′ and 3′ to the open reading frame. In one embodiment, the isolated nucleotide sequence comprises a second promoter operably linked to the one or more RNAi nucleic acid sequences. In one embodiment, the RNAi nucleic acid sequence is about 125 to 500, e.g., about 150 to 175, nucleotides in length. In one embodiment the gene therapy vector may have 2, 3, 4 or more copies of the RNAi nucleic acid sequence which may include miRNA sequences, e.g., miRNA sequences which flank the APOE4 inhibitory sequences.

In one embodiment, a method to prevent, inhibit or treat Alzheimer's disease in a mammal is provided comprising: administering to the mammal an effective amount of a composition comprising the gene therapy vector. In one embodiment, the composition comprises nanoparticles comprising the gene therapy vector or the different vector, or both. In one embodiment, the gene therapy vector or the different vector, or both, comprise a viral vector. In one embodiment, the mammal is a E2/E4 heterozygote. In one embodiment, the mammal is a E4/E4 homozygote. In one embodiment, the composition is systemically administered. In one embodiment, the composition is orally administered. In one embodiment, the composition is intravenously administered. In one embodiment, the composition is locally administered. In one embodiment, the composition is injected. In one embodiment, the composition is administered to the central nervous system. In one embodiment, the composition is administered to the brain. In one embodiment, the composition is a sustained release composition. In one embodiment, the mammal is a human. In one embodiment, the RNAi nucleic acid sequences comprise a plurality of miRNA sequences.

In one embodiment, a method to prevent, inhibit or treat a disease associated with APOE4 expression in a mammal is provided comprising: administering to the mammal an effective amount of a composition comprising the gene therapy vector. In one embodiment, the composition comprises liposomes comprising the gene therapy vector or the different vector, or both. In one embodiment, the composition comprises nanoparticles comprising the gene therapy vector or the different vector, or both. In one embodiment, the gene therapy vector or the different vector, or both, comprise a viral vector. In one embodiment, the mammal is a E2/E4 heterozygote. In one embodiment, the mammal is a E4/E4 homozygote. In one embodiment, the composition is systemically administered. In one embodiment, the composition is orally administered. In one embodiment, the composition is intravenously administered. In one embodiment, the composition is locally administered. In one embodiment, the composition is injected. In one embodiment, the composition is administered to the central nervous system. In one embodiment, the composition is administered to the brain. In one embodiment, the composition is a sustained release composition. In one embodiment, the mammal is a human. In one embodiment, the RNAi sequences comprise a plurality of miRNA sequences.

### BRIEF DESCRIPTION OF THE FIGURES

FIG. 1. Production of inhibitory RNAs from an exemplary target transcript template (Boudreau and Davidson. 2012. Methods in Enzymology, Volume 507).

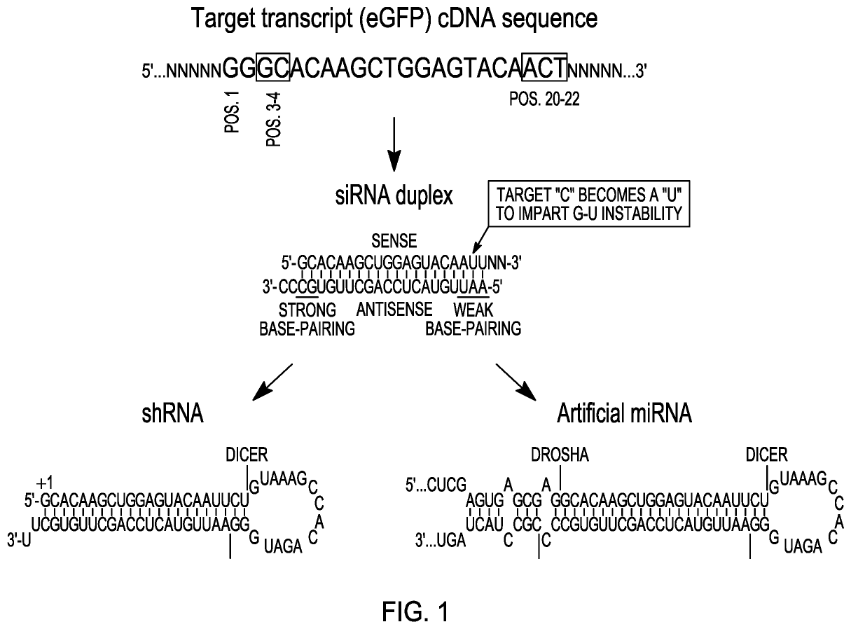

FIG. 2. Pathways to inhibit mRNA (Borel et al., 2014. Mol Ther 22:692-701).

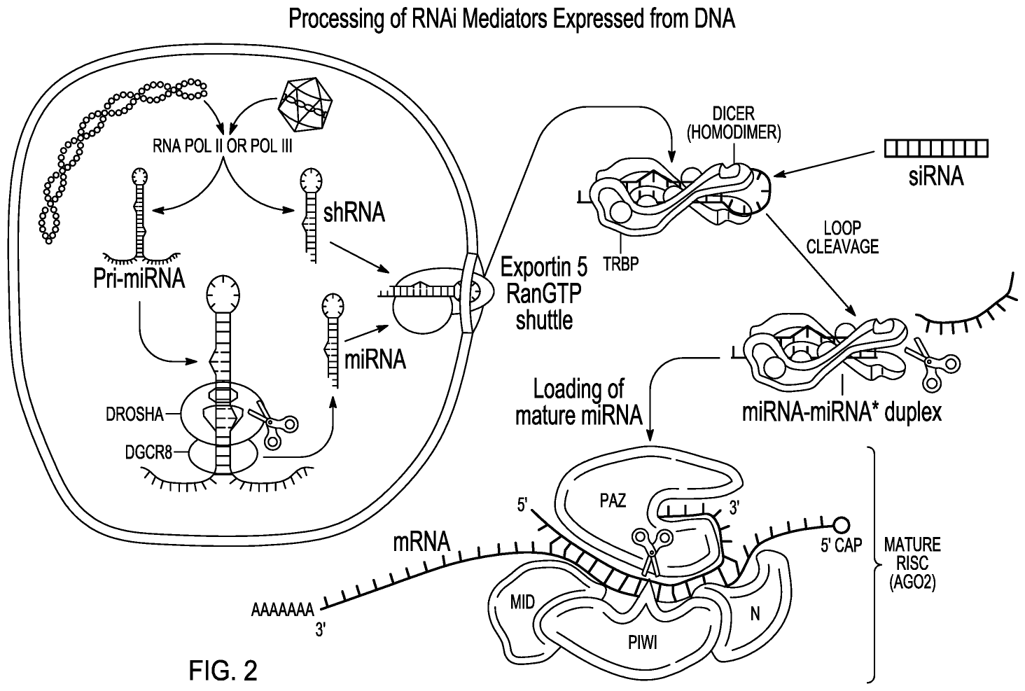

FIG. 3. Exemplary constructs for miRNA insertion(s).

FIG. 4. Single vector and dual vector constructs.

FIG. 5. Single vector construct with two sites for miRNA sequences.

FIG. 6. APOE knock down of expression by four different siRNAs in vitro.

FIG. 7. Use of mir155 scaffold as an exemplary scaffold for miRNA expression.

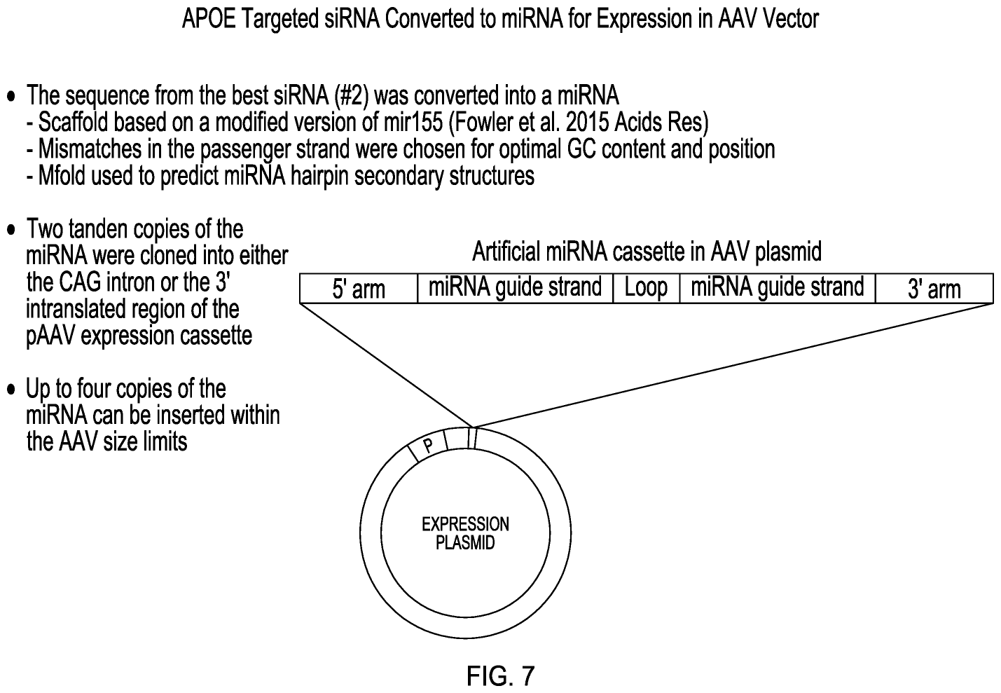

FIG. 8. Mouse experiments.

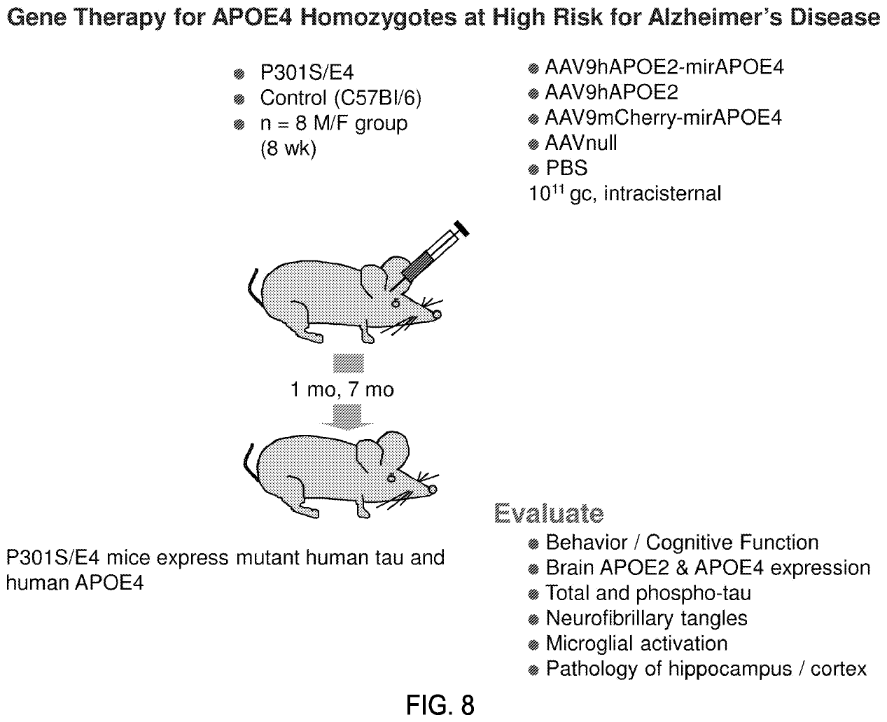

### DETAILED DESCRIPTION

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes: RNA sequences, such as a specific mRNA sequence encoding a specific protein, are found in the cell as a mixture with numerous other mnRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphinylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic.” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GT ATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

Exemplary human APOE sequences include but are not limited to:

(SEQ ID NO: 8)
mkvlwaallv tflagcqakv eqavetepep elrqqtewqs
gqrwelalgr fwdylrwvqt lseqvqeell ssqvtqelra
lmdetmkelk aykseleeql tpvaeetrar lskelqaaqa
rlgadmedvc grlvqyrgev qamlgqstee lrvrlashlr
klrkrllrda ddlqkrlavy qagaregaer glsairerlg
plveqgrvra atvgslagqp lgeraqawge rlrarmeemg
srtrdrldev keqvaevrak leegaqqirl qaeafgarlk
swfeplvedm qrqwaglvek vgaavgtsaa pvpsdnh
(includes signal peptide, italicized above),

as well as sequences with at least 80%, 85%, 90%, 95% or more, e.g., 99% or 100%, sequence identity thereto, including those with Cys at residue 112 (mature polypeptide numbering; c bolded above) and Cys at residue 158 (r bolded above) (APOE2) corresponding to SEQ ID NO:9, or with Arg at residue 112 (mature polypeptide numbering) and Arg at residue 158 (APOE4), corresponding to SEQ ID NO:10, where in one embodiment, APOE4 may have 31K, 46P, 79T, 130R, 163C, 2921H and/or 314R, and APOE2 may have 43C, 152Q, 154C/S, 163C/P, 164Q, 172A, 176C, 242Q, 246C, 254E.

SEQ ID NO: 9 includes
kv eqavetepep elrqqtewqs gqrwelalgr fwdylrwvqt
lseqvgeell ssqvtqelra lmdetmkelk aykseleeql
tpvaeetrar lskelqaaqa rlgadmedvc grlvqyrgev
qamlgqstee lrvrlashlr klrkrllrda ddlqkclavy
qagaregaer glsairerlg plveqgrvra atvgslagqp
lqeraqawge rlrarmeemg srtrdrldev keqvaevrak
leeqaqqirl qaeafqarlk swfeplvedm qrqwaglvek
vqaavgtsaa pvpsdnh.
SEQ ID NO: 10 includes
kv eqavetepep elrqqtewqs gqrwelalgr fwdylrwvqt
lseqvqeell ssqvtqelra lmdetmkelk aykseleeql
tpvaeetrar lskelqaaqa rlgadmedvr grlvqyrgev
qamlgqstee lrvrlashlr klrkrllrda ddlqkrlavy
qagaregaer glsairerlg plveqgrvra atvgslagqp
lqeragawge rlrarmeemg srtrdrldev keqvaevrak
leeqaqgirl qaeafqarlk swfeplvedm qrgwaglvek
vqaavgtsaa pvpsdnh.

Exemplary human APOE nucleic acid sequences, e.g., those for silent nucleotide substitutions if they encode APOE2, include but are not limited to:

(SEQ ID NO: 11)
ggaacttgat gctcagagag gacaagtcat ttgcccaagg
tcacacagct ggcaactggc agagccagga ttcacgccct
ggcaatttga ctccagaatc ctaaccttaa cccagaagca
cggcttcaag cccctggaaa ccacaatacc tgtggcagcc
agggggaggt gctggaatct catttcacat gtggggaggg
ggctcccctg tgctcaaggt cacaaccaaa gaggaagctg
tgattaaaac ccaggtccca tttgcaaagc ctcgactttt
agcaggtgca tcatactgtt cccacccctc ccatcccact
tctgtccagc cgcctagccc cactttcttt tttttctttt
tttgagacag tctccctctt gctgaggctg gagtgcagtg
gcgagatctc ggctcactgt aacctccgcc tcccgggttc
aagcgattct cctgcctcag cctcccaagt agctaggatt
acaggcgccc gccaccacgc ctggctaact tttgtatttt
tagtagagat ggggtttcac catgttggcc aggctggtct
caaactcctg accttaagtg attcgcccac tgtggcctcc
caaagtgctg ggattacagg cgtgagctac cgcccccagc
ccctcccatc ccacttctgt ccagccccct agccctactt
tctttctggg atccaggagt ccagatcccc agccccctct
ccagattaca ttcatccagg cacaggaaag gacagggtca
ggaaaggagg actctgggcg gcagcctcca cattcccctt
ccacgcttgg cccccagaat ggaggagggt gtctggatta
ctgggcgagg tgtcctccct tcctggggac tgtggggggt
ggtcaaaaga cctctatgcc ccacctcctt cctccctctg
ccctgctgtg cctggggcag ggggagaaca gcccacctcg
tgactggggg ctggcccagc ccgccctatc cctgggggag
ggggcgggac agggggagcc ctataattgg acaagtctgg
gatccttgag tcctactcag ccccagcgga ggtgaaggac
gtccttcccc aggagccg
or
(SEQ ID NO: 12)
ccccagcgga ggtgaaggac gtccttcccc aggagccgac
tggccaatca caggcaggaa gatgaaggtt ctgtgggctg
cgttgctggt cacattcctg gcaggatgcc aggccaaggt
ggagcaagcg gtggagacag agccggagcc cgagctgcgc
cagcagaccg agtggcagag cggccagcgc tgggaactgg
cactgggtcg cttttgggat tacctgcgct gggtgcagac
actgtctgag caggtgcagg aggagctgct cagctcccaa
gtcacccaag aactgagggc gctgatggac gagaccatga
aggagttgaa ggcctacaaa tcggaactgg aggaacaact
gaccccggta gcggaggaga cgcgggcacg gctgtccaag
gagctgcaga cggcgcaggc ccggctgggc gcggacatgg
aggacgtgtg cggccgcctg gtgcagtacc gcggcgaggt
gcaggccatg ctcggccaga gcaccgagga gctgcgggtg
cgcctcgcct cccacctgcg caagctgcgt aagcggctcc
tccgcgatcc cgatgacctg cagaagcgcc tggcagtgta
ccaggccggg gcccgcgagg gcgccgagcg cggcctcagc
gccatccgcg agcgcctggg gcccctggtg gaacagggcc
gcgtgcgggc cgccactgtg ggctccctgg ccggccagcc
gctacaggag cgggcccagg cctggggcga gcggctgcgc
gcgcggatgg aggagatggg cagtcggacc cgcgaccgcc
tggacgaggt gaaggagcag gtggcggagg tgcgcgccaa
gctggaggag caggcccagc agatacgcct gcaggccgag
gccttccagg cccgcctcaa gagctggttc gagcccctgg
tggaagacat gcagcgccag tgggccgggc tggtggagaa
ggtgcaggct gccgtgggca ccagcgccgc ccctgtgccc
agcgacaatc actgaacgcc gaagcctgca gccatgcgac
cccacgccac cccgtgcctc ctgcctccgc gcagcctgca
gcgggagacc ctgtccccgc cccagccgtc ctcctggggt
ggaccctagt ttaataaaga ttcaccaagt ttcacgc,

as well as sequences with at least 40%, 50%, 60%, 70%, 80%, 85%, 90%, 95% or more, e.g., 99% or 100%, sequence identity thereto that encode an APOE.

### Compositions and Methods

Alzheimer's disease (AD) directly affects 5 million Americans and is rapidly in-creasing in prevalence and economic impact. Existing drugs have little impact on the underlying disease process and no preventive therapies are currently available. Inheritance of the variant APOE4 gene conveys a high risk for the development of AD, while inheritance of the APOE2 gene is protective, reducing the risk of developing AD) by about 50% and delaying the age of onset. APOE4 is associated with increased brain amyloid load and greater memory impairment in AD. Conversely, APOE2 attenuates these effects. In humans, the odds ratio of developing AD with E4/E4 homozygous genotype is 14.9 and is reduced to 2.6 in E2/E4 heterozygotes. APOE4 may be associated with abnormal brain function apart from its role in promoting amyloid production.

The present disclosure provides for a gene therapy vector for expression of APOE2, sequences to inhibit APOE4 expression, and methods of using the APOE2 and APOE4 inhibitory sequences.

### Exemplary Gene Therapy

The disclosure provides a gene therapy vector comprising a nucleic acid sequence which encodes APOE2 and may include inhibitory sequences of endogenous APOE4 expression, or in one embodiment may include another vector for expression of the inhibitory sequences or a composition having the inhibitory RNA sequences. Various aspects of the gene therapy vector(s) and method are discussed below. Although each parameter is discussed separately, the gene therapy vector and method comprise combinations of the parameters set forth below, e.g., to evoke protection against an APOE4 associated pathology. Accordingly, any combination of parameters can be used according to the gene therapy vector and the method.

A “gene therapy vector” is thus any molecule or composition that has the ability to carry a heterologous nucleic acid sequence into a suitable host cell where synthesis of the encoded protein takes place. Typically, a gene therapy vector is a nucleic acid molecule that has been engineered, using recombinant DNA techniques that are known in the art, to incorporate the heterologous nucleic acid sequence, e.g., heterologous with respect to the other vector sequences such as the promoter or vector backbone sequences such as viral sequences. Desirably, the gene therapy vector is comprised of DNA. Examples of suitable DNA-based gene therapy vectors include plasmids and viral vectors. However, gene therapy vectors that are not based solely on nucleic acids, such as liposomes or nanoparticles, may also be employed. The gene therapy vector can be based on a single type of nucleic acid (e.g., a plasmid) or include non-nucleic acid molecules (e.g., a lipid or a polymer). The gene therapy vector can be integrated into the host cell genome, or can be present in the host cell in the form of an episome.

Gene or siRNA delivery vectors within the scope of the disclosure include, but are not limited to, isolated nucleic acid, e.g., plasmid-based vectors which may be extrachromosomally maintained, and viral vectors. e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes or natural or synthetic polymers. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch.

In one embodiment, the gene therapy vector or the other vector is a viral vector. Suitable viral vectors include, for example, retroviral vectors, lentivirus vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratorv Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells. Adenoviral vectors have been shown to result in transient expression of therapeutic genes in vivo, peaking at 7 days and lasting approximately 4 weeks. In addition, adenoviral vectors can be produced at very high titers, allowing efficient gene therapy with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells.

AAV vectors include but are not limited to AAV1, AAV2, AAV5, AAV7, AAV8, AAV9 or AAVrh10, including chimeric viruses where the AAV genome is from a different source than the capsid.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Exemplary AAV Vectors

In an embodiment, the disclosure provides an adeno-associated virus (AAV) vector which comprises, consists essentially of, or consists of a nucleic acid sequence encoding APOE2. When the AAV vector consists essentially of a nucleic acid sequence encoding APOE2, additional components can be included that do not materially affect the AAV vector (e.g., genetic elements such as poly(A) sequences or restriction enzyme sites that facilitate manipulation of the vector in vitro). When the AAV vector consists of a nucleic acid sequence which encodes APOE2, the AAV vector does not comprise any additional components (i.e., components that are not endogenous to AAV and are not required to effect expression of the nucleic acid sequence).

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61:447 (1990)). These proteins also regulate transcription from endogenous AAV promoters and promoters within helper viruses (see, e.g., Pereira et al., J. Virol., 71:1079 (1997)). The other Rep proteins modify the function of Rep78 and Rep68. The cap genes encode the capsid proteins VP1, VP2, and VP3. The cap genes are transcribed from the p40 promoter.

The AAV vector may be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-5 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, Hum. Gene Ther., 16:541 (2005); and Wu et al., supra). Genome sequences of various AAV serotypes and comparisons thereof are disclosed in, for example, GenBank Accession numbers U89790. J01901, AF043303, and AF085716; Chiorini et al., J. Virol., 71:6823 (1997); Srivastava et al., J. Virol., 45:555 (1983): Chiorini et al., J. Virol., 73:1309 (1999); Rutledge et al., J. Virol., 72:309 (1998); and Wu et al., J. Virol., 74:8635 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et aL, J. Virol., 73(2):939 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (e.g., functionally substitute) corresponding sequences from other serotypes during production of AAV particles in mammalian cells.

Generally, the cap proteins, which determine the cellular tropicity of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., (1):1 (2006); Gao et al., J. Virol., 78:6381 (2004); Gao et al., Proc. Natl. Acad. Sci. USA, 99:11854 (2002); De et al., Mol. Ther., 13:67 (2006); and Gao et al., Mol. Ther., 13:77 (2006).

In one embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). In one embodiment, the AAV vector is generated using an AAV that infects a non-human primate pseudotyped with an AAV that infects humans. Examples of such pseudotyped AAV vectors are disclosed in, e.g., Cearley et al., Molecular Therapy, 13:528 (2006). In one embodiment, an AAV vector can be generated which comprises a capsid protein from an AAV that infects rhesus macaques pseudotyped with AAV2 inverted terminal repeats (ITRs). In a particular embodiment, the AAV vector comprises a capsid protein from AAV10 (also referred to as “AAVrh.10”), which infects rhesus macaques pseudotyped with AAV2 ITRs (see, e.g., Watanabe et al., Gene Ther., 17(8):1042 (2010); and Mao et al., Hum. Gene Therapy, 22:1525 (2011)).

In addition to the nucleic acid sequence encoding APOE2, the AAV vector may comprise expression control sequences, such as promoters, enhancers, polyadenylation signals, transcription terminators, internal ribosome entry sites (IRES), and the like, that provide for the expression of the nucleic acid sequence in a host cell, as well as, in one embodiment, sequences for APOE4 RNAi. Exemplary expression control sequences are known in the art and described in, for example, Goeddel, Gene Expression Technology: Methods in Enzymology, Vol. 185, Academic Press, San Diego, CA. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93:3346 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27:4324 (1999); Nuc. Acid. Res., 2:6e99 (2000); U.S. Pat. No. 7,112,715; and Kramer & Fussenegger, Methods Mol. Biol., 308:123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. In one embodiment, the nucleic acid sequence encoding APOE2, is operably linked to a CMV enhancer/chicken beta-actin promoter (also referred to as a “CAG promoter”) (see, e.g., Niwa et al., Gene, 108:193 (1991); Daly et al., Proc. Nat. Acad. Sci. U.S.A., 96:2296 (1999); and Sondhi et al., Mol. Ther., 15:481 (2007)).

Typically AAV vectors are produced using well characterized plasmids. For example, human embryonic kidney 293T cells are transfected with one of the transgene specific plasmids and another plasmid containing the adenovirus helper and AAV rep and cap genes (specific to AAVrh.10, 8 or 9 as required). After 72 hours, the cells are harvested and the vector is released from the cells by five freeze/thaw cycles. Subsequent centrifugation and benzonase treatment removes cellular debris and unencapsulated DNA. Iodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1× phosphate buffered saline. The viral titers may be measured by TaqMan® real-time PCR and the viral purity may be assessed by SDS-PAGE.

### Pharmaceutical Compositions and Delivery of the Vectors

The disclosure provides a composition comprising, consisting essentially of, or consisting of the above-described gene therapy vector and a pharmaceutically acceptable (e.g., physiologically acceptable) carrier, or a vector for expression of RNAi. When the composition consists essentially of the gene therapy vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the gene therapy vector and the pharmaceutically acceptable carrier, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene therapy vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene therapy vector is administered in a composition formulated to protect the gene therapy vector from damage prior to administration. For example, the composition can be formulated to reduce loss of the gene therapy vector on devices used to prepare, store, or administer the gene therapy vector, such as glassware, syringes, or needles. The composition can be formulated to decrease the light sensitivity and/or temperature sensitivity of the gene therapy vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene therapy vector, facilitate administration, and increase the efficiency of the method. Formulations for gene therapy vector-containing compositions are further described in, for example, Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the gene therapy vector can be present in a composition with other therapeutic or biologically-active agents. For example, factors that control inflammation, such as ibuprofen or steroids, can be part of the composition to reduce swelling and inflammation associated with in vivo administration of the gene therapy vector. Immune system stimulators or adjuvants, e.g., interleukins, lipopolysaccharide, and double-stranded RNA, can be administered to enhance or modify an immune response. Antibiotics, i.e., microbicides and fungicides, can be present to treat existing infection and/or reduce the risk of future infection, such as infection associated with gene therapy procedures.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butyric acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see. e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the gene therapy vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the gene therapy vectors may be intracerebral (including but not limited to intraparenchymal, intraventricular, or intracisternal), intrathecal (including but not limited to lumbar or cisterna magna), or systemic, including but not limited to intravenous, or any combination thereof, using devices known in the art. Delivery may also be via surgical implantation of an implanted device.

The dose of the gene therapy vector in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the gene therapy vector described herein. A “therapeutically effective amount” refers to an amount effective, at dosages and for periods of time necessary, to achieve a desired therapeutic result. “The therapeutically effective amount” may vary according to factors such as the extent of pathology, age, sex, and weight of the individual, and the ability of the gene therapy vector to elicit a desired response in the individual. The dose of gene therapy vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene therapy vector dose range to treat a patient having a particular disease or disorder, based on these and other factors that are well known in the art. The therapeutically effective amount may be between 1×10¹⁰ g genome copies to 1×10³ genome copies. The therapeutically effective amount may be between 1×10¹¹ genome copies to 1×10¹⁴ genome copies. The therapeutically effective amount may be between 1×10¹² genome copies to 1×10¹⁵ genome copies. The therapeutically effective amount may be from 1×10¹³ genome copies (gc) to 1×10¹⁶ gc, e.g., from 1×10¹³ gc to 1×10¹⁴ gc, 1×10¹⁴ gc to 1×10¹⁵ gc, or 1×10¹⁵ gc to 1×10¹⁴ gc. Assuming a 70 kg human, the dose ranges may be from 1.4×10⁸ gc/kg to 1.4×10¹¹ gc/kg, 1.4×10⁹ gc/kg to 1.4×10¹² gc/kg, 1.4×10¹⁰ gc/kg to 1.4×10¹³ gc/kg, or 1.4×10¹¹ gc/kg to 1.4×10¹⁴ gc/kg.

In one embodiment, the composition is administered once to the mammal. It is believed that a single administration of the composition will result in expression of APOE2, and suppression of APOE4 expression, in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure thus provides pharmaceutically acceptable compositions which comprise a therapeutically-effective amount of gene therapy vector comprising a nucleic acid sequence which encodes an APOE2 and a sequence which inhibits APOE4 expression.

### Subjects

The subject may be any animal, including a human and non-human animal. Non-human animals include all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats.

In one embodiment, subjects include human subjects suffering from or at risk for the medical diseases and disorders described herein. The subject is generally diagnosed with the condition by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods may be more appropriate for some ethnic populations such as Caucasians, especially northern European populations, as well as Asian populations.

The term subject also includes subjects of any genotype or phenotype as long as they are in need of treatment, as described above. In addition, the subject can have the genotype or phenotype for any hair color, eye color, skin color or any combination thereof. The term subject includes a subject of any body height, body weight, or any organ or body part size or shape.

### Exemplary Nanoparticle Formulations

Biodegradable nanoparticles, e.g. comprising the gene therapy vector or isolated nucleic acid or a vector for RNAi expression, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride (PSA), poly(carboxybiscarhoxyphenoxyphenoxy hexone (PCPP) poly[bis(p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18: and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The biodegradable nanoparticles may be prepared by methods known in the art. (See. e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, the nanoparticles have a mean effective diameter of less than 1 micron, e.g., the nanoparticles have a mean effective diameter of between about 25 nm and about 500 nm, e.g., between about 50 nm and about 250 nm, about 100 nm to about 150 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA). X-Ray Diffraction (XRD), Aerosol Time of Flight Mass Spectroscopy (ATFMS), and Aerosol Particle Mass Analyzer (APM).

The biodegradable nanoparticles may have a zeta-potential that facilitates uptake by a target cell. Typically, the nanoparticles have a zeta-potential greater than 0. In some embodiments, the nanoparticles have a zeta-potential between about 5 mV to about 45 mV, between about 15 in to about 35 mV, or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a non-viral delivery vehicle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protanine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI). Polyamidoamine (PAMAM), PEI-PEG, PEI-PEG-mannose, dextran-PEI. OVA conjugate, PLGA microparticles, or PLGA microparticles coated with PAMAM, or any combination thereof. The disclosed cationic polymer may include, but are not limited to, polyamidoamine (PAMAM) dendrimers, Polyamidoamine dendrimers suitable for preparing the presently disclosed nanoparticles may include 3rd-, 4th-, 5th-, or at least 6th-generation dendrimers.

In one embodiment, the delivery vehicle comprises a lipid, e.g., N-[1-(2,3-dioleoyloxy)propel]-N,N,N-trimethylammonium (DOTMA), 2,3-dioleyloxy-N-[2-spermine carboxamide]ethyl-N,N-dimethyl-1-propanaminium trifluoracetate (DOSPA, Lipofectamine); 1,2-dioleoyl-3-trimethylammonium-propane (DOTAP); N-[1-(2,3-dimyristoyl) propyl]; N,N-dimethyl-N-(2-hydroxyethyl) ammonium bromide (DMRIE), 3-β-[N—(N,N-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amnines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamner), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcelluloses, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(ε-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis(p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range of materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isopropylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

### EXEMPLARY EMBODIMENTS

In one embodiment, a gene therapy vector is provided comprising a promoter operably linked to a nucleic acid sequence comprising an open reading frame encoding APOE2 and a 3′ untranslated region (3′ UTR), and a nucleotide sequence having RNAi sequences corresponding to APOE4 for inhibition of APOE4 mRNA. In one embodiment, the vector comprises the nucleotide sequence. In one embodiment, the nucleotide sequence is 5′ or 3′ to the open reading frame. In one embodiment, the nucleotide sequence is 5′ and 3′ to the open reading frame. In one embodiment, the nucleotide sequence is on a different vector. In one embodiment, the vector is a viral vector. In one embodiment, the viral vector is an AAV, adenovirus, lentivirus, herpesvirus or retrovirus vector. In one embodiment, the AAV is AAV5, AAV9 or AAVrh10. In one embodiment, the APOE4 is human APOE4. In one embodiment, the APOE2 is human APOE2. In one embodiment, the nucleotide sequence is linked to a second promoter. In one embodiment,

the second promoter is a PolIII promoter. In one embodiment, the RNAi comprises miRNA including a plurality of miRNA sequences. In one embodiment, the RNAi comprises siRNA including a plurality of siRNA sequences. In one embodiment, the open reading frame comprises a plurality of silent nucleotide substitutions. In one embodiment, at least 5%, 10%, 20%, 30%, 40%, 50%, 60%, 70%, 80% or 90% of the codons have a silent nucleotide substitution. In one embodiment, the open reading frame further comprises a peptide tag. In one embodiment, the tag comprises HA, histidine tag, AviTag, maltose binding tag, Strep-tag, FLAG-tag, V5-tag, Myc-tag, Spot-tag, T7 tag, or NE-tag.

Also provided is a host cell or mammal comprising the vector. In one embodiment, the cell is a mammalian cell. In one embodiment, the cell is a human cell. In one embodiment, the mammal is a non-human primate. In one embodiment, the mammal is a human.

Further provided is a method to prevent, inhibit or treat Alzheimer's disease in a mammal, comprising: administering to the mammal an effective amount of a composition comprising the gene therapy vector.

A method to prevent, inhibit or treat a disease associated with APOE4 expression in a mammal is provided comprising: administering to the mammal an effective amount of a composition comprising the gene therapy vector.

In one embodiment, a composition comprises liposomes comprising the vector. In one embodiment, the composition comprises nanoparticles comprising the nucleic acid. In one embodiment, the gene therapy vector comprises a viral vector. In one embodiment, the mammal is a E2/E4 heterozygote. In one embodiment, the mammal is a E4/E4 homozygote. In one embodiment, the composition is systemically administered. In one embodiment, the composition is orally administered. In one embodiment, the composition is intravenously administered. In one embodiment, the composition is locally administered. In one embodiment, the composition is injected. In one embodiment, the composition is administered to the central nervous system. In one embodiment, the composition is administered to the brain. In one embodiment, the composition is a sustained release composition. In one embodiment, the mammal is a human. In one embodiment, the RNAi sequences comprise a plurality of miRNA sequences, e.g., identical miRNA sequences.

The invention will be described by the following non-limiting examples.

### Example 1

Alzheimer's disease (AD) affects 5 million Americans and is rapidly increasing in prevalence. Existing drugs have little impact on underlying disease processes and no preventative therapies are currently available. Inheritance of the APOE4 allele represents a high risk for development of disease while inheritance of the APOE2 allele is protective, reducing the risk of developing AD by >50% and delaying age of onset. Adeno-associated virus (AAV) delivery of the human APOE2 gene to murine models of AD expressing human APOE4 (homozygous expression) demonstrated reduced amyloid-β peptide and amyloid burden. The odds ratio of developing AD is reduced in E2/E4 heterozygotes compared with E4/E4 homozygotes (2.6 vs. 14.9). Suppression of APOE4, e.g., via delivery of an AAV vector, while simultaneously expressing human APOE2 may reduce the risk for AD even further. In one embodiment, gene therapy such as AAV therapy is designed to deliver both the human APOE2 gene coding sequence and artificial RNAs such as microRNA(s) (miRNA) targeted to the endogenous APOE4. The combination of knockdown of detrimental endogenous APOE4 expression with the expression of the beneficial APOE2 allele may provide enhanced protection from AD development for individuals with homozygous for the APOE4 allele.

In one embodiment, siRNA interacts with mRNA to silence translation. To express an siRNA from a DNA sequence, such as a gene therapy expression vector, the targeting sequence must be embedded in a small hairpin RNA (shRNA) or miRNA scaffold. The vector-expressed artificial miRNAs are similar to endogenous RNAi's and undergo two processing steps. Since miRNAs are expressed at lower levels they are less likely to induce liver and CNS toxicity upon delivery by gene therapy vectors.

In one embodiment, knockdown with miRNA against all isoforms of endogenous APOE may be accomplished using multiple miRNAs targeting different sections of APOE mRNA, thereby enhancing silencing. In one embodiment, vector-derived human APOE2 may contain silent mutations in the coding sequence to prevent silencing.

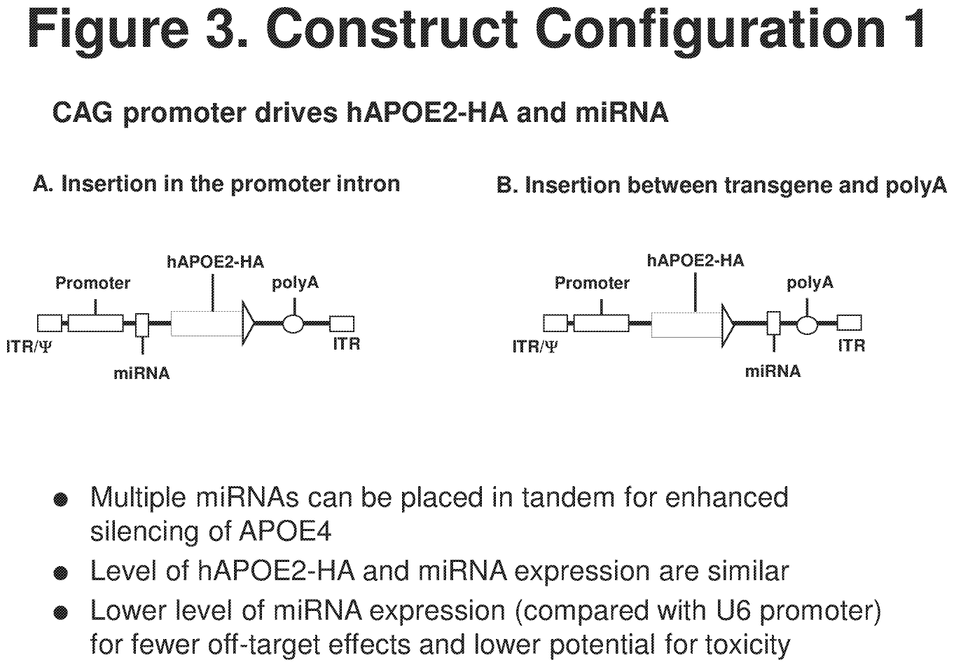

As shown in FIG. 3, the miRNA having the RNAi sequences to inhibit APOE4 expression may be inserted into 5′ non-coding sequences, e.g., an intron, and/or 3′ non-coding sequences. Multiple miRNAs can be placed in tandem for enhanced silencing of, e.g., APOE4. It was found that the level of hAPOE2-HA and miRNA expression were similar. There was a lower level of miRNA expression (compared with an U6 promoter) which in turn results in fewer off-target effects and lower potential for toxicity.

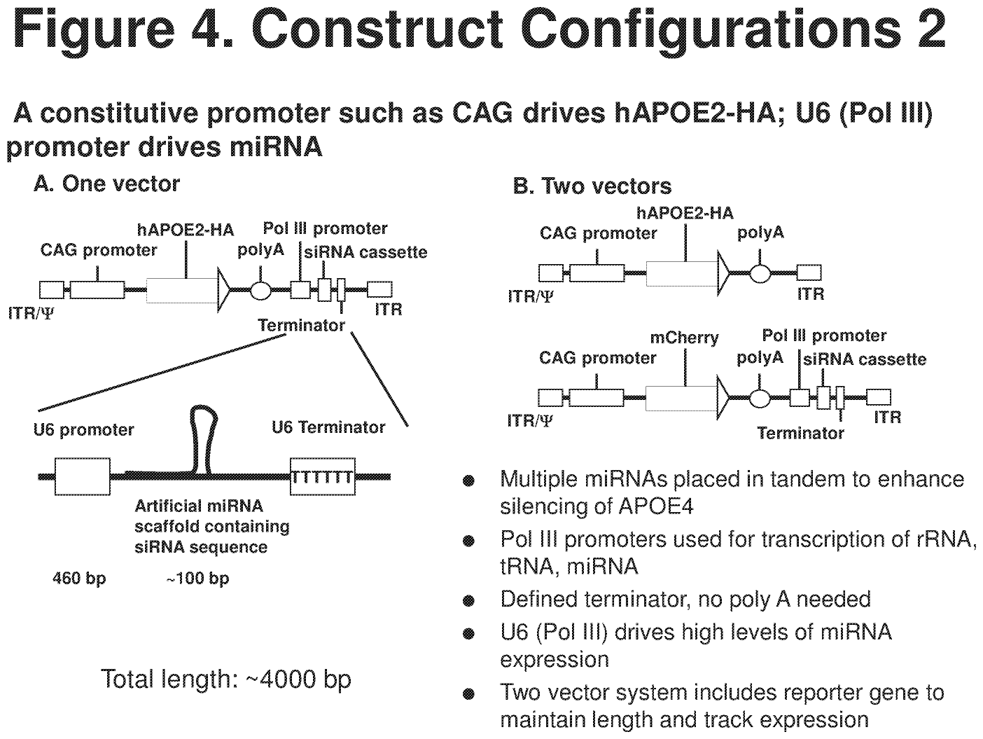

In one embodiment (see FIG. 4), a constitutive promoter such as CAG drives hAPOE2-HA and an U6 promoter (an exemplary Pol III promoter) promoter drives miRNA. In one embodiment, multiple miRNAs are placed in tandem to enhance silencing of APOE4, e.g., 2, 3 4 or more miRNAs. In one embodiment. Pol III promoters are used for transcription of rRNA, tRNA, and/or miRNA. In one embodiment, a vector may have a defined terminator, e.g., no poly A is needed since PolIII transcription is terminated by an oligo (dT) stretch in the non-template strand (dA in the template strand)). In one embodiment, a two vector system may be employed where the second vector includes a stuffer sequence, e.g., for a reporter gene, to maintain length and track expression.

Thus, the disclosure provides for a vector, e.g., a viral vector such as an AAV vector, delivering both the human APOE2 gene and artificial miRNAs targeting human APOE4. These gene therapy vectors can be used to mitigate the risk of AD development in APOE4 homozygous individuals (as well as E2/E4 heterozygotes) by tipping the balance toward the expression of the beneficial APOE2 allele.

In one embodiment, the vector is useful in disorders or diseases which may benefit from increased APOE2 and/or decreased APOE4. In one embodiment, the vector is delivered to a mammal such as human at risk of AD. AD currently affects 5 million people in the US and worldwide prevalence is expected to rise to 65 million by 2030. Global prevalence of the APOE4 allele is 15% and about 50% of AD patients carry at least one APOE4 allele. Detrimental APOE4 gene is targeted for decreased expression while protective APOE2 expression is provided and the risk related to APOE4 is further reduced compared to a gene therapy that only delivers APOE2.

### Example 2

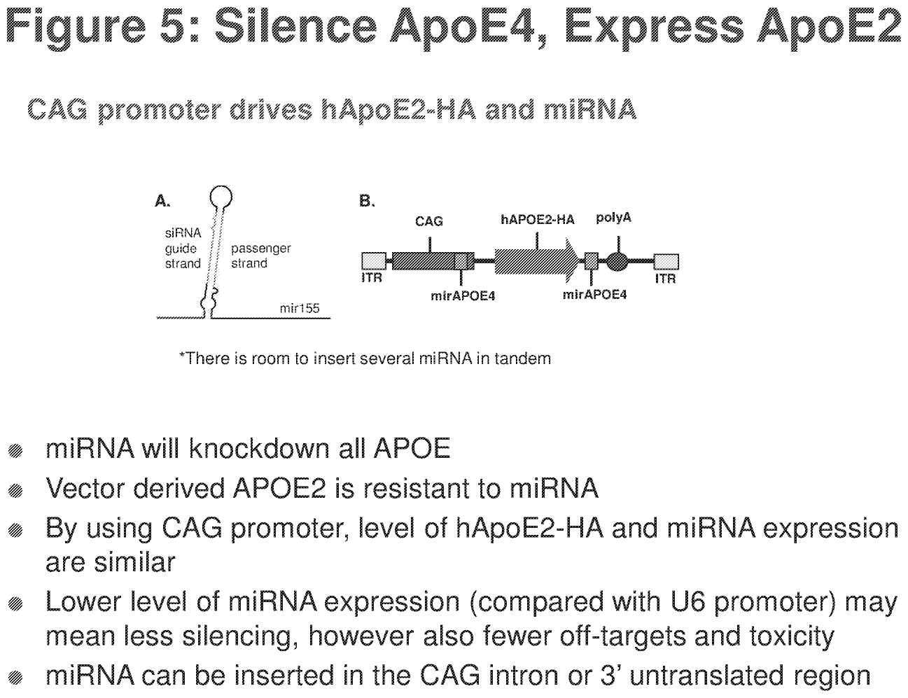

FIG. 5 shows a system where miRNA knocks down all APOE isoform expression and where the vector derived APOE2 is resistant to miRNA. For example, by using a CAG promoter, the level of hApoE2-HA and miRNA expression were similar and a lower level of miRNA expression (compared with U6 promoter) may mean less silencing, however, there are also fewer off-targets and toxicity. In one embodiment, miRNA can be inserted in the CAG intron or 3′ untranslated region.

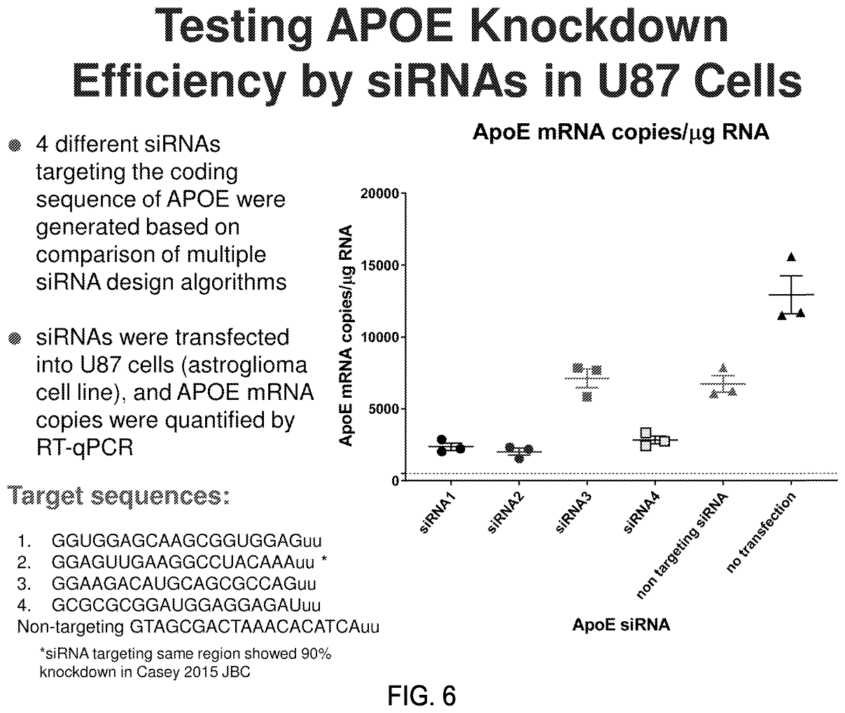

FIG. 6 depicts testing of APOE knockdown efficiency by siRNAs in U87 cells. Four different siRNAs targeting the coding sequence of APOE were generated based on a comparison of multiple siRNA design algorithms, siRNAs were transfected into U87 cells (astroglioma cell line), and APOE nmRNA copies were quantified by RT-qPCR. The identified sequences were as follows:

(SEQ ID NO: 1)
1. GGUGGAGCAAGCGGUGGAGuu
(SEQ ID NO: 2)
2. GGAGUUGAAGGCCUACAAAuu
(SEQ ID NO: 3)
3. GGAAGACAUGCAGCGCCAGuu
(SEQ ID NO: 4)
4. GCGCGCGGAUGGAGGAGAUuu
(SEQ ID NO: 5)
Non-targeting siRNA is GTAGCGACTAAACACATCAuu

Other sequences for siRNAs include:

(SEQ ID NO: 20)
GCCGATGACCTGCAGAAGCuu
(SEQ ID NO: 21)
GCGCGCGGATGGAGGAGATuu
(SEQ ID NO: 22)
GTAAGCGGCTCCTCCGCGAuu

The sequence from one siRNA (112 above) was converted into a miRNA. A scaffold based on a modified version of mir155 (Fowler et al. 2015 Nucl. Acids Res., 44:e48, the disclosure of which is incorporated by reference herein) was employed. However, any miRNA backbone may be employed, e.g., mir21, mir30 or mir33.

For example, for a miR from siRN-A #2, the following may be employed:

(SEQ ID NO: 23)
CTGGAGGCTTGCTGAAGGCTGTATGCTGATTTGTAGGCCTTCAACTCC
T
GTTTTGGCCACTGACTGACAGGAGTGAGGCCTACAAATCAGGACACA
AGGCCTGTTACTAGCACTCACATGGAACAAATGGCC;
(SEQ ID NO: 24)
CTGGAGGCTTGCTTTGGGCTGTATGCTGATTTGTAGGCCTTCAACTCC
TGTTTTGGCCACTGACTGACAGGAGTTGAAGTCACAAATCAGGACAC
AAGGCCCTTTATCAGCACTCACATGGAACAAATGGCCACCGTGGGAG
GATGACAA;
or
(SEQ ID NO: 25)
CTGGAGGCTTGCTTTGGGCTGTATGCTGTTCCGATTTGTAGGCCTTCA
AGTTTTGGCCACTGACTGACTTGAAGTCACAAATCGGAACAGGACAC
AAGGCCCTTTATCAGCACTCACATGGAACAAATGGCCACCGTGGGAG
GATGACAA

In one embodiment, the miRNA has one or more of: a U or A at guide position 1 relative to the 5′ microprocessor cleavage site, U or A at positions 2-7, 10-14 and 17, and G or C at positions 19-21, and/or C/C content of 36.4% to 45.5%, and/or a guide strand that is 2 nucleotides longer than passenger strand, and/or mismatches in 1) a loop where 3 to 5 adjacent nucleotides of the guide strand are not base paired with target strand, 2) a 3 bp spaced mismatch where 2 single guide strand nucleotide mismatches are separated by 3 guide/passenger base pairs and/or 3) a 4 bp spaced mismatch where 2 single guide strand nucleotide mismatches are separated by 4 guide/passenger base pairs. Mismatches in the passenger strand were chosen for optimal GC content and position. Mfold was used to predict miRNA hairpin secondary structures. Two tandem copies of the miRNA were cloned into either the CAG intron or the 3′ untranslated region of the pAAV expression cassette. Up to four copies of the miRNA (of that length) can be inserted within the AAV size limits.

Vector derived APOE2 was modified to be resistant to silencing by the targeting miRNA mentioned above (see underlined sequence below). Silent changes were made in the nucleotide sequence of the miRNA targeting region (red/bold).

APOE2 from vector:
(SEQ ID NO: 6)
ATGAAGGTTCTGTGGGCTGCGTTGCTGGTCACATTCCTGGCAG
GATGCCAGGCCAAGGTGGAGCAAGCGGTGGAGACAGAGCCGGAGCC
CGAGCTGCGCCAGCAGACCGAGTGGCAGAGCGGCCAGCGCTGGGAA
CTGGCACTGGGTCGCTTTTGGGATTACCTGCGCTGGGTGCAGACACT
GTCTGAGCAGGTGCAGGAGGAGCTGCTCAGCTCCCAGGTCACCCAGG
AACTGAGGGCGCTGATGGACGAGACCATGAAGGAGTTGAAGGCCTA
CAAATCGGAACTGGAGGAACAACTGACCCCGGTGGCGGAGGAGACG
CGGGCACGGCTGTCCAAGGAGCTGCAGGCGGCGCAGGCCCGGCTGG
GCGCGGACATGGAGGACGTGTGCGGCCGCCTGGTGCAGTACCGCGGC
GAGGTGCAGGCCATGCTCGGCCAGAGCACCGAGGAGCTGCGGGTGC
GCCTCGCCTCCCACCTGCGCAAGCTGCGTAAGCGGCTCCTCCGCGAT
GCCGATGACCTGCAGAAGTGCCTGGCAGTGTACCAGGCCGGGGCCCG
CGAGGGCGCCGAGCGCGGCCTCAGCGCCATCCGCGAGCGCCTGGGG
CCCCTGGTGGAACAGGGCCGCGTGCGGGCCGCCACTGTGGGCTCCCT
GGCCGGCCAGCCGCTACAGGAGCGGGCCCAGGCCTGGGGCGAGCGG
CTGCGCGCGCGGATGGAGGAGATGGGCAGCCGGACCCGCGACCGCC
TGGACGAGGTGAAGGAGCAGGTGGCGGAGGTGCGCGCCAAGCTGGA
GGAGCAGGCCCAGCAGATACGCCTGCAGGCCGAGGCCTTCCAGGCCC
GCCTCAAGAGCTGGTTCGAGCCCCTGGTGGAAGACATGCAGCGCCAG
TGGGCCGGGCTGGTGGAGAAGGTGCAGGCTGCCGTGGGCACCAGCG
CCGCCCCTGTGCCCAGCGACAATCAC
Modified APOE2:
(SEQ ID NO: 7)
ATGAAGGTTCTGTGGGCTGCGTTGCTGGTCACATTCCTGGCAG
GATGCCAGGCCAAGGTGGAGCAAGCGGTGGAGACAGAGCCGGAGCC
CGAGCTGCGCCAGCAGACCGAGTGGCAGAGCGGCCAGCGCTGGGAA
CTGGCACTGGGTCGCTTTTGGGATTACCTGCGCTGGGTGCAGACACT
GTCTGAGCAGGTGCAGGAGGAGCTGCTCAGCTCCCAGGTCACCCAGG
AACTGAGGGCGCTGATGGACGAGACCATGAAAGAACTCAAAGCTTA
TAAGAGCGAGCTGGAGGAACAACTGACCCCGGTGGCGGAGGAGACG
CGGGCACGGCTGTCCAAGGAGCTGCAGGCGGCGCAGGCCCGGCTGG
GCGCGGACATGGAGGACGTGTGCGGCCGCCTGGTGCAGTACCGCGGC
GAGGTGCAGGCCATGCTCGGCCAGAGCACCGAGGAGCTGCGGGTGC
GCCTCGCCTCCCACCTGCGCAAGCTGCGTAAGCGGCTCCTCCGCGAT
GCCGATGACCTGCAGAAGTGCCTGGCAGTGTACCAGGCCGGGGCCCG
CGAGGGCGCCGAGCGCGGCCTCAGCGCCATCCGCGAGCGCCTGGGG
CCCCTGGTGGAACAGGGCCGCGTGCGGGCCGCCACTGTGGGCTCCCT
GGCCGGCCAGCCGCTACAGGAGCGGGCCCAGGCCTGGGGCGAGCGG
CTGCGCGCGCGGATGGAGGAGATGGGCAGCCGGACCCGCGACCGCC
TGGACGAGGTGAAGGAGCAGGTGGCGGAGGTGCGCGCCAAGCTGGA
GGAGCAGGCCCAGCAGATACGCCTGCAGGCCGAGGCCTTCCAGGCCC
GCCTCAAGAGCTGGTTCGAGCCCCTGGTGGAAGACATGCAGCGCCAG
TGGGCCGGGCTGGTGGAGAAGGTGCAGGCTGCCGTGGGCACCAGCG
CCGCCCCTGTGCCCAGCGACAATCAC

The sequence above changes all possible nucleotides silently while considering codon usage within the composite recognition site for the 3 miRNAs derived from siRNA #2. However, other examples are shown below:

Modified APOE:
(SEQ ID NO: 26)
ATGAAGGTTCTGTGGGCTGCGTTGCTGGTCACATTCCTGGCAGGATG
CCAGGCCAAGGTGGAGCAAGCGGTGGAGACAGAGCCGGAGCCCGAG
CTGCGCCAGCAGACCGAGTGGCAGAGCGGCCAGCGCTGGGAACTGG
CACTGGGTCGCTTTTGGGATTACCTGCGCTGGGTGCAGACACTGTCTG
AGCAGGTGCAGGAGGAGCTGCTCAGCTCCCAGGTCACCCAGGAACTG
AGGGCGCTGATGGACGAGACCATGAAAGAACTTAAAGCATATAAGA
GTGAGCTGGAGGAACAACTGACCCCGGTGGCGGAGGAGACGCGGGC
ACGGCTGTCCAAGGAGCTGCAGGCGGCGCAGGCCCGGCTGGGCGCG
GACATGGAGGACGTGTGCGGCCGCCTGGTGCAGTACCGCGGCGAGGT
GCAGGCCATGCTCGGCCAGAGCACCGAGGAGCTGCGGGTGCGCCTCG
CCTCCCACCTGCGCAAGCTGCGTAAGCGGCTCCTCCGCGATGCCGAT
GACCTGCAGAAGTGCCTGGCAGTGTACCAGGCCGGGGCCCGCGAGG
GCGCCGAGCGCGGCCTCAGCGCCATCCGCGAGCGCCTGGGGCCCCTG
GTGGAACAGGGCCGCGTGCGGGCCGCCACTGTGGGCTCCCTGGCCGG
CCAGCCGCTACAGGAGCGGGCCCAGGCCTGGGGCGAGCGGCTGCGC
GCGCGGATGGAGGAGATGGGCAGCCGGACCCGCGACCGCCTGGACG
AGGTGAAGGAGCAGGTGGCGGAGGTGCGCGCCAAGCTGGAGGAGCA
GGCCCAGCAGATACGCCTGCAGGCCGAGGCCTTCCAGGCCCGCCTCA
AGAGCTGGTTCGAGCCCCTGGTGGAAGACATGCAGCGCCAGTGGGCC
GGGCTGGTGGAGAAGGTGCAGGCTGCCGTGGGCACCAGCGCCGCCC
CTGTGCCCAGCGACAATCAC;
Modified APOE:
(SEQ ID NO: 27)
ATGAAGGTTCTGTGGGCTGCGTTGCTGGTCACATTCCTGGCAGGATG
CCAGGCCAAGGTGGAGCAAGCGGTGGAGACAGAGCCGGAGCCCGAG
CTGCGCCAGCAGACCGAGTGGCAGAGCGGCCAGCGCTGGGAACTGG
CACTGGGTCGCTTTTGGGATTACCTGCGCTGGGTGCAGACACTGTCTG
AGCAGGTGCAGGAGGAGCTGCTCAGCTCCCAGGTCACCCAGGAACTG
AGGGCGCTGATGGACGAGACCATGAAAGAACTCAAAGCATATAAGA
GTGAGCTGGAGGAACAACTGACCCCGGTGGCGGAGGAGACGCGGGC
ACGGCTGTCCAAGGAGCTGCAGGCGGCGCAGGCCCGGCTGGGCGCG
GACATGGAGGACGTGTGCGGCCGCCTGGTGCAGTACCGCGGCGAGGT
GCAGGCCATGCTCGGCCAGAGCACCGAGGAGCTGCGGGTGCGCCTCG
CCTCCCACCTGCGCAAGCTGCGTAAGCGGCTCCTCCGCGATGCCGAT
GACCTGCAGAAGTGCCTGGCAGTGTACCAGGCCGGGGCCCGCGAGG
GCGCCGAGCGCGGCCTCAGCGCCATCCGCGAGCGCCTGGGGCCCCTG
GTGGAACAGGGCCGCGTGCGGGCCGCCACTGTGGGCTCCCTGGCCGG
CCAGCCGCTACAGGAGCGGGCCCAGGCCTGGGGCGAGCGGCTGCGC
GCGCGGATGGAGGAGATGGGCAGCCGGACCCGCGACCGCCTGGACG
AGGTGAAGGAGCAGGTGGCGGAGGTGCGCGCCAAGCTGGAGGAGCA
GGCCCAGCAGATACGCCTGCAGGCCGAGGCCTTCCAGGCCCGCCTCA
AGAGCTGGTTCGAGCCCCTGGTGGAAGACATGCAGCGCCAGTGGGCC
GGGCTGGTGGAGAAGGTGCAGGCTGCCGTGGGCACCAGCGCCGCCC
CTGTGCCCAGCGACAATCAC;
Modified APOE:
(SEQ ID NO: 28)
ATGAAGGTTCTGTGGGCTGCGTTGCTGGTCACATTCCTGGCAGGATG
CCAGGCCAAGGTGGAGCAAGCGGTGGAGACAGAGCCGGAGCCCGAG
CTGCGCCAGCAGACCGAGTGGCAGAGCGGCCAGCGCTGGGAACTGG
CACTGGGTCGCTTTTGGGATTACCTGCGCTGGGTGCAGACACTGTCTG
AGCAGGTGCAGGAGGAGCTGCTCAGCTCCCAGGTCACCCAGGAACTG
AGGGCGCTGATGGACGAGACCATGAAAGAACTTAAAGCTTATAAGA
GTGAGCTGGAGGAACAACTGACCCCGGTGGCGGAGGAGACGCGGGC
ACGGCTGTCCAAGGAGCTGCAGGCGGCGCAGGCCCGGCTGGGCGCG
GACATGGAGGACGTGTGCGGCCGCCTGGTGCAGTACCGCGGCGAGGT
GCAGGCCATGCTCGGCCAGAGCACCGAGGAGCTGCGGGTGCGCCTCG
CCTCCCACCTGCGCAAGCTGCGTAAGCGGCTCCTCCGCGATGCCGAT
GACCTGCAGAAGTGCCTGGCAGTGTACCAGGCCGGGGCCCGCGAGG
GCGCCGAGCGCGGCCTCAGCGCCATCCGCGAGCGCCTGGGGCCCCTG
GTGGAACAGGGCCGCGTGCGGGCCGCCACTGTGGGCTCCCTGGCCGG
CCAGCCGCTACAGGAGCGGGCCCAGGCCTGGGGCGAGCGGCTGCGC
GCGCGGATGGAGGAGATGGGCAGCCGGACCCGCGACCGCCTGGACG
AGGTGAAGGAGCAGGTGGCGGAGGTGCGCGCCAAGCTGGAGGAGCA
GGCCCAGCAGATACGCCTGCAGGCCGAGGCCTTCCAGGCCCGCCTCA
AGAGCTGGTTCGAGCCCCTGGTGGAAGACATGCAGCGCCAGTGGGCC
GGGCTGGTGGAGAAGGTGCAGGCTGCCGTGGGCACCAGCGCCGCCC
CTGTGCCCAGCGACAATCAC;
Modified APOE:
(SEQ ID NO: 29)
ATGAAGGTTCTGTGGGCTGCGTTGCTGGTCACATTCCTGGCAGGATG
CCAGGCCAAGGTGGAGCAAGCGGTGGAGACAGAGCCGGAGCCCGAG
CTGCGCCAGCAGACCGAGTGGCAGAGCGGCCAGCGCTGGGAACTGG
CACTGGGTCGCTTTTGGGATTACCTGCGCTGGGTGCAGACACTGTCTG
AGCAGGTGCAGGAGGAGCTGCTCAGCTCCCAGGTCACCCAGGAACTG
AGGGCGCTGATGGACGAGACCATGAAAGAACTTAAAGCATATAAGA
GCGAGCTGGAGGAACAACTGACCCCGGTGGCGGAGGAGACGCGGGC
ACGGCTGTCCAAGGAGCTGCAGGCGGCGCAGGCCCGGCTGGGCGCG
GACATGGAGGACGTGTGCGGCCGCCTGGTGCAGTACCGCGGCGAGGT
GCAGGCCATGCTCGGCCAGAGCACCGAGGAGCTGCGGGTGCGCCTCG
CCTCCCACCTGCGCAAGCTGCGTAAGCGGCTCCTCCGCGATGCCGAT
GACCTGCAGAAGTGCCTGGCAGTGTACCAGGCCGGGGCCCGCGAGG
GCGCCGAGCGCGGCCTCAGCGCCATCCGCGAGCGCCTGGGGCCCCTG
GTGGAACAGGGCCGCGTGCGGGCCGCCACTGTGGGCTCCCTGGCCGG
CCAGCCGCTACAGGAGCGGGCCCAGGCCTGGGGCGAGCGGCTGCGC
GCGCGGATGGAGGAGATGGGCAGCCGGACCCGCGACCGCCTGGACG
AGGTGAAGGAGCAGGTGGCGGAGGTGCGCGCCAAGCTGGAGGAGCA
GGCCCAGCAGATACGCCTGCAGGCCGAGGCCTTCCAGGCCCGCCTCA
AGAGCTGGTTCGAGCCCCTGGTGGAAGACATGCAGCGCCAGTGGGCC
GGGCTGGTGGAGAAGGTGCAGGCTGCCGTGGGCACCAGCGCCGCCC
CTGTGCCCAGCGACAATCAC;
Modified APOE:
(SEQ ID NO: 30)
ATGAAGGTTCTGTGGGCTGCGTTGCTGGTCACATTCCTGGCAGGATG
CCAGGCCAAGGTGGAGCAAGCGGTGGAGACAGAGCCGGAGCCCGAG
CTGCGCCAGCAGACCGAGTGGCAGAGCGGCCAGCGCTGGGAACTGG
CACTGGGTCGCTTTTGGGATTACCTGCGCTGGGTGCAGACACTGTCTG
AGCAGGTGCAGGAGGAGCTGCTCAGCTCCCAGGTCACCCAGGAACTG
AGGGCGCTGATGGACGAGACCATGAAAGAACTCAAAGCTTATAAGA
GTGAGCTGGAGGAACAACTGACCCCGGTGGCGGAGGAGACGCGGGC
ACGGCTGTCCAAGGAGCTGCAGGCGGCGCAGGCCCGGCTGGGCGCG
GACATGGAGGACGTGTGCGGCCGCCTGGTGCAGTACCGCGGCGAGGT
GCAGGCCATGCTCGGCCAGAGCACCGAGGAGCTGCGGGTGCGCCTCG
CCTCCCACCTGCGCAAGCTGCGTAAGCGGCTCCTCCGCGATGCCGAT
GACCTGCAGAAGTGCCTGGCAGTGTACCAGGCCGGGGCCCGCGAGG
GCGCCGAGCGCGGCCTCAGCGCCATCCGCGAGCGCCTGGGGCCCCTG
GTGGAACAGGGCCGCGTGCGGGCCGCCACTGTGGGCTCCCTGGCCGG
CCAGCCGCTACAGGAGCGGGCCCAGGCCTGGGGCGAGCGGCTGCGC
GCGCGGATGGAGGAGATGGGCAGCCGGACCCGCGACCGCCTGGACG
AGGTGAAGGAGCAGGTGGCGGAGGTGCGCGCCAAGCTGGAGGAGCA
GGCCCAGCAGATACGCCTGCAGGCCGAGGCCTTCCAGGCCCGCCTCA
AGAGCTGGTTCGAGCCCCTGGTGGAAGACATGCAGCGCCAGTGGGCC
GGGCTGGTGGAGAAGGTGCAGGCTGCCGTGGGCACCAGCGCCGCCC
CTGTGCCCAGCGACAATCAC
Modified APOE:
(SEQ ID NO: 31)
ATGAAGGTTCTGTGGGCTGCGTTGCTGGTCACATTCCTGGCAGGATG
CCAGGCCAAGGTGGAGCAAGCGGTGGAGACAGAGCCGGAGCCCGAG
CTGCGCCAGCAGACCGAGTGGCAGAGCGGCCAGCGCTGGGAACTGG
CACTGGGTCGCTTTTGGGATTACCTGCGCTGGGTGCAGACACTGTCTG
AGCAGGTGCAGGAGGAGCTGCTCAGCTCCCAGGTCACCCAGGAACTG
AGGGCGCTGATGGACGAGACCATGAAAGAACTCAAAGCATATAAGA
GCGAGCTGGAGGAACAACTGACCCCGGTGGCGGAGGAGACGCGGGC
ACGGCTGTCCAAGGAGCTGCAGGCGGCGCAGGCCCGGCTGGGCGCG
GACATGGAGGACGTGTGCGGCCGCCTGGTGCAGTACCGCGGCGAGGT
GCAGGCCATGCTCGGCCAGAGCACCGAGGAGCTGCGGGTGCGCCTCG
CCTCCCACCTGCGCAAGCTGCGTAAGCGGCTCCTCCGCGATGCCGAT
GACCTGCAGAAGTGCCTGGCAGTGTACCAGGCCGGGGCCCGCGAGG
GCGCCGAGCGCGGCCTCAGCGCCATCCGCGAGCGCCTGGGGCCCCTG
GTGGAACAGGGCCGCGTGCGGGCCGCCACTGTGGGCTCCCTGGCCGG
CCAGCCGCTACAGGAGCGGGCCCAGGCCTGGGGCGAGCGGCTGCGC
GCGCGGATGGAGGAGATGGGCAGCCGGACCCGCGACCGCCTGGACG
AGGTGAAGGAGCAGGTGGCGGAGGTGCGCGCCAAGCTGGAGGAGCA
GGCCCAGCAGATACGCCTGCAGGCCGAGGCCTTCCAGGCCCGCCTCA
AGAGCTGGTTCGAGCCCCTGGTGGAAGACATGCAGCGCCAGTGGGCC
GGGCTGGTGGAGAAGGTGCAGGCTGCCGTGGGCACCAGCGCCGCCC
CTGTGCCCAGCGACAATCAC;
or
Modified APOE:
(SEQ ID NO: 32)
ATGAAGGTTCTGTGGGCTGCGTTGCTGGTCACATTCCTGGCAGGATG
CCAGGCCAAGGTGGAGCAAGCGGTGGAGACAGAGCCGGAGCCCGAG
CTGCGCCAGCAGACCGAGTGGCAGAGCGGCCAGCGCTGGGAACTGG
CACTGGGTCGCTTTTGGGATTACCTGCGCTGGGTGCAGACACTGTCTG
AGCAGGTGCAGGAGGAGCTGCTCAGCTCCCAGGTCACCCAGGAACTG
AGGGCGCTGATGGACGAGACCATGAAAGAACTTAAAGCTTATAAGA
GCGAGCTGGAGGAACAACTGACCCCGGTGGCGGAGGAGACGCGGGC
ACGGCTGTCCAAGGAGCTGCAGGCGGCGCAGGCCCGGCTGGGCGCG
GACATGGAGGACGTGTGCGGCCGCCTGGTGCAGTACCGCGGCGAGGT
GCAGGCCATGCTCGGCCAGAGCACCGAGGAGCTGCGGGTGCGCCTCG
CCTCCCACCTGCGCAAGCTGCGTAAGCGGCTCCTCCGCGATGCCGAT
GACCTGCAGAAGTGCCTGGCAGTGTACCAGGCCGGGGCCCGCGAGG
GCGCCGAGCGCGGCCTCAGCGCCATCCGCGAGCGCCTGGGGCCCCTG
GTGGAACAGGGCCGCGTGCGGGCCGCCACTGTGGGCTCCCTGGCCGG
CCAGCCGCTACAGGAGCGGGCCCAGGCCTGGGGCGAGCGGCTGCGC
GCGCGGATGGAGGAGATGGGCAGCCGGACCCGCGACCGCCTGGACG
AGGTGAAGGAGCAGGTGGCGGAGGTGCGCGCCAAGCTGGAGGAGCA
GGCCCAGCAGATACGCCTGCAGGCCGAGGCCTTCCAGGCCCGCCTCA
AGAGCTGGTTCGAGCCCCTGGTGGAAGACATGCAGCGCCAGTGGGCC
GGGCTGGTGGAGAAGGTGCAGGCTGCCGTGGGCACCAGCGCCGCCC
CTGTGCCCAGCGACAATCAC.

Vectors may be tested in non-human animals such as mice. In one embodiment, the vector includes sequences from the AAV9-CAG-APOE2 vector (AAV9-APOE2), the adeno-associated viral vector serotype 9 expressing the APOE2 behind the chicken actin promoter or from the AAVrh.10-CAG-APOE2 vector (AAVrh.10-APOE2), the rhesus adeno-associated viral vector serotype 10 expressing the APOE2 transgene behind the chickenβ actin promoter.

The AAVrh.10 and AAV9 vectors may be produced and purified as described previously (Sondhi et al., 2007, 2012; Zolotukhin et al., 2002). Briefly, the vectors are produced by cotransfection of HEK293T cells with an expression cassette plasmid and adenoviral helper plasmids. The packaging cell line, HEK293T, is maintained in Dulbecco's modified Eagles medium, supplemented with 5% fetal bovine serum, 100 U/mL penicillin, 100 mg/mL streptomycin, and maintained at 37° C. with 5% CO₂. The cells are plated at 30%-40% confluence in CellSTACKS (Corning. Tewksbury, MA) for 24 hours (or when at 70%-80% confluence) followed by transfection with plasmids using the PEIpro procedure. The cells are incubated at 37° C. for 3 days before harvesting and lysing by 5 freeze/thaw cycles. The resulting cell lysate is treated with 50 U/mL of Benzonase at 37° C. for 30 minutes. For AAVrh.10 vectors, the cell lysate is purified by iodixanol density gradient followed by Q-HP ion-exchange chromatography. For AAV9 vectors, the cell lysate is precipitated in PEG (final concentration of PEG: 8%) overnight. After centrifugation, the supernatant is discarded and the pellet is resuspended in 15 mL lysis buffer (150 mM NaCl, 50 mM Tris-1Cl, pH 8.5). The sample is purified by centrifugation through 1.37 g/mL CsCl in a 38.5 mL polyallomer tube using an SW28 rotor at 24,000 rpm (182,000 g), at 20° C. for 24 hours. A 21-gauge needle (Hamilton, Reno. NV) is inserted through the bottom side of the centrifuge tube and 1 mL fractions are collected. Vector-containing fractions are determined by dot blot with a ³²P-labeled probe containing the sequence of the vector constructs. The positive fractions are subsequently pooled and diluted with 1.37 g/mL CsCl, and the samples are loaded into a 13.5 mL Quick-Seal tube and centrifuged in an ultra-centrifuge (Beckman LE-80K; Beckman Coulter, Fullerton, CA) 90 Ti rotor, at 67,000 rpm (384,000 g), 20° C. for 16-20 hours. Fractions (0.5 mL) are collected, and the positive fractions were pooled. The purified AAVrh.10 or AAV9 vectors are concentrated in phosphate-buffered saline (PBS). Vector genome titer is determined by Tag-Man quantitative polymerase chain reaction. The purified vectors are sterile filtered; tested 14 days for growth on medium supporting the growth of aerobic bacteria, anaerobic bacteria, or fungi; tested for endotoxin; and demonstrated to be mycoplasma free.

The AAV preparation (2 mL, 1.0×10¹⁰ vg or at another dose) may be injected using, e.g., a 33-gauge needle (Hamilton) and a syringe pump (KD Scientific, Holliston, MA) at a rate of 0.2 mL/min.

### REFERENCES

Baek et al., PLoS One, 5:e13468 (2010).
Bales et al., J. Neurosci., 29:6771 (2009).
Cearley and Wolfe, J. Neurosci., 27:9928 (2007).
Bales et al., Nat. Genet., 17:263e264 (1997).
Boyles et al., J. Clin. Invest., 76:1501e1513 (1985).
Carrasquillo et al., Neurobiol. Aging. 36:60e67 (2015).
Castellano et al., Sci. Transl. Med., 3:89ra57 (2011).
Corder et al., Nat. Genet., 7:180e184 (1994).
Corder et al., Science, 261:921e923 (1993).
Deane et al., J. Clin. Invest., 118:4002e4013 (2008).
DeMattos et al., Neurochem. Int., 39:415e425 (2001).
DiBattista et al., Curr. Alzheimer Res., 13: 1200 (2016).
Dodart et al., Proc. Natl. Acad. Sci. U.S.A., 102:1211e1216 (2005).
Fagan et al., Neurobiol. Dis., 9:305e318 (2002).
Fan et al., Biofactors, 35:239e248 (2009).
Farrer et al., JAMA, 278:1349e1356 (1997).
Franklin and Paxinos, The mouse brain in Stereotaxic Coordinates, 3rd edition. Elsevier Inc, New York (2007).
Games et al., Nature, 373:523e527 (1995).
Haass and Selkoe, Nat. Rev. Mol. Cell Biol., 8:101e112 (2007).
Hardy and Selkoe, Science, 297:353e356 (2002).
Hashimoto et al., J. Neurosci., 32:151 S1e15192 (2012).
Hatters et al., Trends Biochem. Sci., 31:445e454 (2006).
Heffernan A et al., J. Mol. Neurosci., 60:316 (2016).
Holtzman et al., Proc. Natl. Acad. Sci. U.S.A., 97:2892e2897 (2000).
Holtzman et al., Cold Spring Harb. Perspect. Med., 2:a006312 (2012).
Hudry et al., Sci. Transl. Med., 5:212ra161 (2013).
Johnson-Wood et al., Proc. Natl. Acad. Sci. U.S.A., 94:1550e1555 (1997).
Kells et al., Proc. Natl. Acad. Sci. U.S.A., 106:2407e2411 (2009).
Kim et al., Neuron, 63:287e303 (2009).
Kim et al., J. Neurosci., 31:18007e18012 (2011).
Lambert et al., Nat. Genet., 45:1452e1458 (2013).
Lemere and Masliah, Nat. Rev. Neurol., 6:108e119 (2010).
Li et al., J. Bio. Chem., 287:44593e44601 (2012).
Liu et al., Nat. Rev. Neurol., 9:106e118 (2013).
Manelli et al., J. Mol. Neurosci., 23:235e246 (2004).
Morris et al., Ann. Neurol., 67:122e131 (2010).
Ramanan et al., Mol. Psychiatry, 19:351e357 (2013).
Rebeck et al., Neuron, 11:575e580 (1993).
Reiman et al., Proc. Natl. Acad. Sci. U.S.A., 106:6820e6825 (2009).
Rosenberg et al., Hum. Gene Ther. Clin. Dev., 29:24 (2018).
Safieb et al., BMC Medicine, 17:64 (2019).
Saunders et al., Neurogy, 43:1467e1472 (1993).
Schmechel et al., Proc. Natl. Acad. Sci. U.S.A, 90:9649e9653 (1993).
Schrnued and Hopkins, Brain Res., 874:123 (2000).
Sondhi et al., Mol. Ther., 15:481e491 (2007).
Sondhi et al., Hum. Gene Ther. Method, 23:324e335 (2012).
Strittmatter et al., Proc. Natl. Acad. Sci. U.S.A., 90:1977e1981 (1993).
Sullivan et al., Neurobiol. Aging, 32:791e801 (2011).
Sullivan et al., J. Biol. Chem., 272:17972e17980 (1997).
Suri et al., Neurosci. Biobehav. Rev., 37:2878e2886 (2013).
Talbot et al., Lancet, 343:1432e1433 (1994).
Tsirka et al., Proc. Natl. Acad. Sci., 94:9779 (1997).
Walker et al., Acta Neuropathol., 100:36e42 (2000).
Youmans et al., J. Biol. Chem., 287:41774e41786 (2012).
Yu et al., Annu. Rev. Neurosci., 37:79e100 (2014).
Zhao et al., J. Neurosci., 29:3603e3612 (2009).
Zhao et al., Neurobiol Aging, 44:159 (2016).
Zolotukhin et al., Methods, 28:158e167 (2002).

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A gene therapy vector comprising a first promoter operably linked to a nucleic acid sequence comprising an open reading frame encoding APOE2 and a 3′ untranslated region, and an isolated nucleotide sequence comprising one or more RNAi nucleic acid sequences for inhibition of APOE4 mRNA.

2. The vector of claim 1 wherein the vector comprises the nucleotide sequence.

3-5. (canceled)

6. The vector of claim 1 wherein the isolated nucleotide sequence comprises a second promoter operably linked to the one or more RNAi nucleic acid sequences.

7. The vector of claim 1 wherein the gene therapy vector is a viral vector.

8-10. (canceled)

11. The vector of claim 1 wherein the APOE4 is human APOE4 or wherein the APOE2 is human APOE2.

12. (canceled)

13. The vector of claim 1 wherein the first promoter is a PolI promoter.

14. (canceled)

15. The vector of claim 1 wherein the isolated nucleotide sequence comprises nucleic acid for one or more miRNAs comprising two or more of the RNAi nucleic acid sequences or wherein the RNAi comprises siRNA including a plurality of siRNA sequences.

16. (canceled)

17. The vector of claim 1 wherein the open reading frame for APOE2 comprises a plurality of silent nucleotide substitutions relative to SEQ ID NO:6.

18. The vector of claim 17 wherein the plurality of the silent nucleotide substitutions in the APOE2 open reading frame are not in the RNAi nucleic acid sequence in the isolated nucleotide sequence.

19-20. (canceled)

21. The vector of claim 1 wherein the APOE4 that is inhibited has a sequence having at least 80%, 85%, 90%, 95% or more amino acid sequence identity to a polypeptide comprising SEQ ID NO: 10.

22. (canceled)

23. The vector of claim 1 wherein the one or more RNAi nucleic acid sequences have at least 60%, 70%, 80% 90% or more nucleotide sequence identity to one of SEQ ID Nos. 1-4 or the complement thereof.

24. The vector of claim 1 comprising a first promoter operably linked to a nucleic acid sequence comprising an open reading frame encoding human APOE2 and an isolated nucleotide sequence having one or more RNAi nucleic acid sequences for inhibition of human APOE4 mRNA.

25-27. (canceled)

28. The vector of claim 24 wherein the isolated nucleotide sequence comprises a second promoter operably linked to the one or more RNAi nucleic acid sequences.

29-30. (canceled)

31. A method to prevent, inhibit or treat a disease associated with APOE4 expression in a mammal, comprising: administering to the mammal an effective amount of a composition comprising the gene therapy vector of claim 1.

32-34. (canceled)

35. The method of claim 31 wherein the mammal is a E2/E4 heterozygote or homozygote.

36-41. (canceled)

42. The method of claim 31 wherein the composition is administered to the central nervous system.

43-44. (canceled)

45. The method of claim 31 wherein the mammal is a human.

46. The method of claim 31 wherein the RNAi sequences comprise a plurality of miRNA sequences each comprising the one or more RNAi nucleic acid sequences for inhibition of APOE4 mRNA.

47. The method of claim 46 wherein one of the miRNA sequences in the vector is inserted 5′ to the open reading frame and another is inserted 3′ to the open reading frame.

48. The method of claim 31 wherein the RNAi sequences comprise a miRNA sequence comprising the one or more RNAi nucleic acid sequences for inhibition of APOE4 mRNA.

49-52. (canceled)


SEQUENCE LISTING (32 sequences)

SEQ ID NO: 1 - RNA, 21 nt, Artificial Sequence
  A synthetic oligonucleotide
gguggagcaagcgguggaguu

SEQ ID NO: 2 - RNA, 21 nt, Artificial Sequence
  A synthetic oligonucleotide
ggaguugaaggccuacaaauu

SEQ ID NO: 3 - RNA, 21 nt, Artificial Sequence
  A synthetic oligonucleotide
ggaagacaugcagcgccaguu

SEQ ID NO: 4 - RNA, 21 nt, Artificial Sequence
  A synthetic oligonucleotide
gcgcgcggauggaggagauuu

SEQ ID NO: 5 - RNA, 21 nt, Artificial Sequence
  A synthetic oligonucleotide
gtagcgactaaacacatcauu

SEQ ID NO: 6 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 7 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagcgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 8 - protein, 317 aa, Homo sapiens [sequence omitted]

SEQ ID NO: 9 - protein, 299 aa, Artificial Sequence
  A synthetic polypeptide
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 10 - protein, 299 aa, Artificial Sequence
  A synthetic polypeptide
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 11 - DNA, 1098 bp, Homo sapiens
ggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctggattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccg

SEQ ID NO: 12 - DNA, 1157 bp, Homo sapiens
ccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaagtcacccaagaactgagggcgctgatggacgagaccatgaaggagttgaaggcctacaaatcggaactggaggaacaactgaccccggtagcggaggagacgcgggcacggctgtccaaggagctgcagacggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatcccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagtcggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 13 - DNA, 35 bp, Artificial Sequence
  A synthetic oligonucleotide
gaaagaactcaaagcttataagagcgagctggagg

SEQ ID NO: 14 - RNA, 22 nt, Artificial Sequence
  A synthetic oligonucleotide
  misc_feature (1)...(22): n = A,T,C or G
gcacaagcuggaguacaauunn

SEQ ID NO: 15 - RNA, 22 nt, Artificial Sequence
  A synthetic oligonucleotide
aauuguacuccagcuugugccc

SEQ ID NO: 16 - RNA, 60 nt, Artificial Sequence
  A synthetic oligonucleotide
gcacaagcuggaguacaauucuguaaagccacagaugggaauuguacuccagcuugugcu

SEQ ID NO: 17 - RNA, 84 nt, Artificial Sequence
  A synthetic oligonucleotide
cucgaguggcgggcacaagcuggaguacaauucuguaaagccacagaugggaauuguacuccagcuugugccccgccuacuagu

SEQ ID NO: 18 - DNA, 32 bp, Artificial Sequence
  A synthetic oligonucleotide
  misc_feature (1)...(32): n = A,T,C or G
nnnnngggcacaagctggagtacaactnnnnn

SEQ ID NO: 19 - skipped

SEQ ID NO: 20 - DNA, 21 bp, Artificial Sequence
  A synthetic oligonucleotide
gccgatgacctgcagaagcuu

SEQ ID NO: 21 - DNA, 21 bp, Artificial Sequence
  A synthetic oligonucleotide
gcgcgcggatggaggagatuu

SEQ ID NO: 22 - DNA, 21 bp, Artificial Sequence
  A synthetic oligonucleotide
gtaagcggctcctccgcgauu

SEQ ID NO: 23 - DNA, 132 bp, Artificial Sequence
  A synthetic oligonucleotide
ctggaggcttgctgaaggctgtatgctgatttgtaggccttcaactcctgttttggccactgactgacaggagtgaggcctacaaatcaggacacaaggcctgttactagcactcacatggaacaaatggcc

SEQ ID NO: 24 - DNA, 150 bp, Artificial Sequence
  A synthetic oligonucleotide
ctggaggcttgctttgggctgtatgctgatttgtaggccttcaactcctgttttggccactgactgacaggagttgaagtcacaaatcaggacacaaggccctttatcagcactcacatggaacaaatggccaccgtgggaggatgacaa

SEQ ID NO: 25 - DNA, 150 bp, Artificial Sequence
  A synthetic oligonucleotide
ctggaggcttgctttgggctgtatgctgttccgatttgtaggccttcaagttttggccactgactgacttgaagtcacaaatcggaacaggacacaaggccctttatcagcactcacatggaacaaatggccaccgtgggaggatgacaa

SEQ ID NO: 26 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaacttaaagcatataagagtgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 27 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcatataagagtgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 28 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaacttaaagcttataagagtgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 29 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaacttaaagcatataagagcgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 30 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcttataagagtgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 31 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaactcaaagcatataagagcgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 32 - DNA, 951 bp, Artificial Sequence
  A synthetic oligonucleotide
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaagaacttaaagcttataagagcgagctggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac